(19) United States
(12) Patent Application Publication
(10) Pub. No.: US 2025/0177769 A1
(43) Pub. Date: Jun. 5, 2025

(54) DEVICE FOR DEEP TRANSCRANIAL MAGNETIC STIMULATION REGULATION TREATMENT

(71) Applicant: Xuanwu Hospital, Capital Medical University, Beijing (CN)

(72) Inventor: Yuping WANG, Beijing (CN)

(73) Assignee: Xuanwu Hospital, Capital Medical University, Beijing (CN)

(21) Appl. No.: 18/670,348

(22) Filed: May 21, 2024

(30) Foreign Application Priority Data
Dec. 5, 2023 (CN) 202311647965.5

(51) Int. Cl. A61N 2/00; A61N 2/02

(52) U.S. Cl. CPC A61N 2/006; A61N 2/02

(57) ABSTRACT

A device for deep transcranial magnetic stimulation regulation treatment is provided. The device includes an upper computer control software, configured to send a stimulation instruction to a system main control module according to predetermined parameter data; a system main control module, configured to send a control enable signal to a stimulation coil excitation power module when receiving the stimulation instruction; a stimulation coil excitation power module, configured to provide a current to a deep stimulation coil module according to the control enable signal; and a deep stimulation coil module, configured to generate a superimposed magnetic field and transmit the superimposed magnetic field to a target region. The deep stimulation coil module includes multiple coil groups. Each coil group includes two coils. There is an overlapping part among magnetic fields of the multiple coil groups, and the overlapping part corresponds to a predetermined target region.

### CROSS-REFERENCE TO RELATED APPLICATION

This patent application claims the benefit and priority of Chinese Patent Application No. 202311647965.5 filed with the China National Intellectual Property Administration on Dec. 5, 2023, the disclosure of which is incorporated by reference herein in its entirety as part of the present application.

### BACKGROUND OF THE INVENTION

### Field of the Invention

At least one embodiment relates to the field of medical devices, and in particular to a device for deep transcranial magnetic stimulation regulation treatment.

### Description of the Related Art

The technology for transcranial magnetic stimulation (TMS) is a noninvasive human brain stimulation technology, which can regulate the function of nerve tissues by providing repetitive pulsed magnetic fields. Numerous clinical studies have proved the effectiveness and safety of TMS in treating brain functional diseases. Existing magnetic stimulation modules include annular coils, “8”-shaped coils, biconical coils, and H-shaped coils, etc. However, the usually stimulation depth is 2-3 cm, and the deepest does not exceed 6 cm (H-shaped coils). Therefore, the existing magnetic stimulation methods cannot effectively reach the deep tissues of the human brain, such as temporal lobe, basal ganglia, thalamus, etc., but can only be used for the regulation and treatment of superficial sites of the brain, leading to the limitation on clinical application.

At present, the neural regulation technologies capable of reaching deep brain tissues such as temporal lobe, basal ganglia and thalamus are all invasive technologies, such as deep brain stimulation (DBS), which are expensive and incapable of adjusting target points once implanted. There is no noninvasive neural regulation technology capable of replacing DBS.

### BRIEF SUMMARY OF THE INVENTION

An objective of at least one embodiment of the disclosure is to provide a device for deep transcranial magnetic stimulation regulation treatment, which makes a non-invasive transcranial magnetic field accurately reach a deep brain region by generating a superimposed electromagnetic field.

To achieve the objective above, at least one embodiment of the disclosure employs the following technical solution.

A device for deep transcranial magnetic stimulation regulation treatment includes an upper computer control software, a system main control module, a stimulation coil excitation power module, and a deep stimulation coil module.

An output end of the upper computer control software is connected to an input end of the system main control module, an output end of the system main control module is connected to a control end of the stimulation coil excitation power module, and an output end of the stimulation coil excitation power module is connected to an input end of the deep stimulation coil module.

The upper computer control software is configured to send a stimulation instruction to the system main control module according to predetermined parameter data, where the predetermined parameter data includes a target region, excitation time, and an excitation current.

The system main control module is configured to send a control enable signal to the stimulation coil excitation power module when receiving the stimulation instruction.

The stimulation coil excitation power module is configured to provide a current to the deep stimulation coil module according to the control enable signal.

The deep stimulation coil module is configured to generate a superimposed magnetic field and transmit the superimposed magnetic field to the target region.

The deep stimulation coil module includes multiple coil groups. Each coil group includes two coils. There is an overlapping part among magnetic fields of the plurality of coil groups, and the overlapping part corresponds to a predetermined target region.

According to the specific embodiments provided by the disclosure, the disclosure has the following technical effects: a superimposed electromagnetic field generated by controlling a deep stimulation coil module is transmitted to a target region, thus realizing that a non-invasive transcranial magnetic field accurately reaches a deep brain region.

### BRIEF DESCRIPTION OF THE DRAWINGS

To describe the technical solutions of the embodiments of the disclosure or in the prior art more clearly, the following briefly introduces the accompanying drawings required for describing the embodiments. Apparently, the accompanying drawings in the following description show merely some embodiments of the disclosure, and those of ordinarily skilled in the art may still derive other drawings from these accompanying drawings without creative efforts.

FIG. 1 is a structural diagram of a device for deep transcranial magnetic stimulation regulation treatment according to an embodiment of the disclosure; and

FIG. 2 is a structural diagram of a stimulation module according to an embodiment of the disclosure.

### DETAILED DESCRIPTION OF THE INVENTION

The following clearly and completely describes the technical solutions in the embodiments of the disclosure with reference to the accompanying drawings in the embodiments of the disclosure. Apparently, the described embodiments are merely a part rather than all of the embodiments of the disclosure. All other embodiments obtained by a person of ordinarily skilled in the art based on the embodiments of the disclosure without creative efforts shall fall within the protection scope of the disclosure.

It is expected that the non-invasive transcranial magnetic field can accurately reaches intracranial cavity, so as to generate an induced current and biological effect in a target region without damaging nerve tissues in the region where the magnetic field passes. It is expected that multiple magnetic stimulation modules can focus on the same target point in the deep brain from different directions (similar to the principle of radiotherapy), and cumulative magnetic field on each path is far less than that of the focused target point, and has weak effect, and it is only at the target point that the induced current and magnetic field of physiological effect are generated. Through the registration of individualized brain magnetic resonance structural images, stimulation parameters before stimulation can be stimulated by designing the software, thus predicting a target-point position and physiological effect under the conditions of a stimulation magnetic field approach and stimulation parameters.

An objective of the disclosure is to provide a device for deep transcranial magnetic stimulation regulation treatment, which makes a non-invasive transcranial magnetic field accurately reach a deep brain region by generating a superimposed electromagnetic field.

In order to make the objectives, technical solutions and advantages of the disclosure more clearly, the disclosure is further described in detail below with reference to the accompanying drawings and the embodiments.

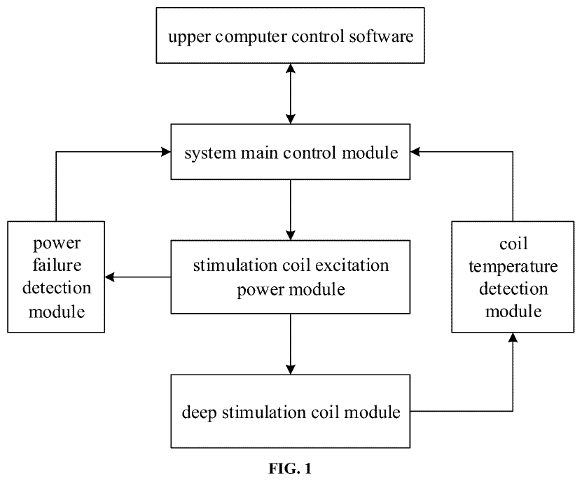

As shown in FIG. 1, a device for deep transcranial magnetic stimulation regulation treatment includes an upper computer control software, a system main control module, a stimulation coil excitation power module, and a deep stimulation coil module.

An output end of the upper computer control software is connected to an input end of the system main control module, an output end of the system main control module is connected to a control end of the stimulation coil excitation power module, and an output end of the stimulation coil excitation power module is connected to an input end of the deep stimulation coil module.

The upper computer control software is configured to send a stimulation instruction to the system main control module according to predetermined parameter data. The predetermined parameter data includes a target region, excitation time, and an excitation current.

During specific implementation, the target region is obtained by a doctor through the examination of a patient, and the stimulation parameters such as excitation time and excitation current are obtained by the doctor according to the treatment regulation scheme, which is not the focus of the disclosure, and thus will not be described in detail here.

The system main control module is configured to send a control enable signal to the stimulation coil excitation power module when receiving the stimulation instruction.

The stimulation coil excitation power module is configured to provide a current to the deep stimulation coil module according to the control enable signal.

The deep stimulation coil module is configured to generate a superimposed magnetic field and transmit the superimposed magnetic field to the target region.

The deep stimulation coil module includes multiple coil groups, and each coil group includes two coils. There is an overlapping part among magnetic fields of the multiple coil groups, and the overlapping part corresponds to a predetermined target region.

During specific implementation, a simulation experiment is performed according to an obtained position of the target region, so as to determine position data of each coil group. Each coil group is adjusted according to the position data of each coil group, there is an overlapping part between the magnetic fields generated by the multiple coil groups, and the overlapping part corresponds to an obtained position of the target region. After the adjustment of the multiple coil groups is completed, the target region is stimulated by the device for deep transcranial magnetic stimulation regulation treatment provided by the disclosure. The position data includes three-dimensional coordinates and/or angular data.

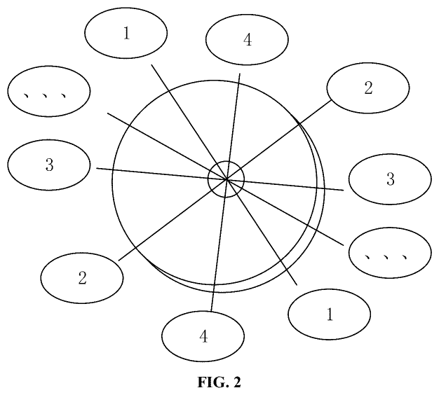

The same number in FIG. 2 denotes one coil group, a current flowing through each coil group has the same magnitude and direction. The multiple coil groups are placed in different orientations of the brain, and the superimposed electromagnetic field generated by each coil group in a certain target region of the brain is used to regulate and stimulate this region. The positions and angles of the coil groups can be adjusted according to different target regions (the target region is an existing established region in the medical field), so that the generated electromagnetic fields are superimposed in the target region, and the multiple coil groups are added for greater intensity of regulatory stimulation. The use of the multiple coil groups also reduces requirements on a coil excitation power supply, improves power usage efficiency, and reduces safety risks.

In some embodiments, the device for deep transcranial magnetic stimulation regulation treatment further includes a coil temperature detection module.

The coil temperature detection module is connected to the system main control module, and the coil temperature detection module is located inside the deep stimulation coil module.

The coil temperature detection module is configured to collect a temperature of the deep stimulation coil module in real time, and to transmit the temperature to the system main control module. The system main control module is configured to control the on/off of the stimulation coil excitation power module according to the temperature. The system main control module transmits the temperature to the upper computer control software. The upper computer control software is configured to send a stop instruction to the system main control module when the temperature is greater than a predetermined temperature threshold, and the upper computer control software displays temperature failure information.

The deep stimulation coil module, after being powered on, generates an electromagnetic field required for regulation, so as to achieve the stimulation on a brain region. Simultaneously, the coil temperature detection module is configured to detect the temperature of the deep stimulation coil module in real time, and to send the temperature to the system main control module. The system main control module can achieve the control (on/off) on the stimulation coil excitation power module through hardware. The system main control module further sends the temperature to the upper computer control software, and the upper computer control software performs threshold comparison according to the temperature, and sends a stop instruction to the system main control module when the temperature is greater than the predetermined temperature threshold. The upper computer control software displays temperature failure information to prompt failure.

The device for deep transcranial magnetic stimulation regulation treatment further includes a power failure detection module.

The power failure detection module is connected to the system main control module and the stimulation coil excitation power module.

The power failure detection module is configured to collect an operating state of the stimulation coil excitation power module in real time, and to transmit the operating state to the system main control module. The system main control module is configured to control the on/off of the stimulation coil excitation power module according to the operating state. The system main control module is also configured to transmit an off state of the stimulation coil excitation power module to the upper computer control software when controlling the stimulation coil excitation power module to be turned off. The upper computer control software displays the off state of the stimulation coil excitation power module, and sends a stop instruction to the system main control module.

The stimulation coil excitation power module is configured to provide an operating current for the deep stimulation coil module according to the signal sent by the system main control module. Simultaneously, the power failure detection module is configured to detect an operating state of the stimulation coil excitation power module, and to send an abnormal state signal to the system main control module when detecting the abnormal state signal (a power switching signal is detected, if the detected switching signal is 0, it is determined that the stimulation coil excitation power module is in an abnormal state, and if the detected switching signal is 1, it is determined that the stimulation coil excitation power module is in a normal state). The system main control module is used to achieve the control (on/off) of the stimulation coil excitation power module according to the signal sent by the power failure detection module.

According to the disclosure, a non-invasive transcranial magnetic field can accurately reach a deep brain region by generating the superimposed electromagnetic field. The deep brain region is a brain region with a depth greater than 6 cm.

Various embodiments in this specification are described in a progressive way, and each embodiment focuses on the differences from other embodiments, so the same or similar parts in each embodiment can be referred to each other.

Specific examples are used herein for illustration of the principles and implementation methods of the disclosure. The description of the embodiments is merely used to help illustrate the method and its core principles of the disclosure. In addition, those of ordinarily skilled in the art can make various modifications in terms of specific embodiments and scope of application in accordance with the teachings of the disclosure. In conclusion, the content of this specification shall not be construed as a limitation to the disclosure.

1. A device for deep transcranial magnetic stimulation regulation treatment, comprising:
an upper computer control software,
a system main control module,
a stimulation coil excitation power module, and
a deep stimulation coil module;
wherein
an output end of the upper computer control software is connected to an input end of the system main control module, an output end of the system main control module is connected to a control end of the stimulation coil excitation power module, and an output end of the stimulation coil excitation power module is connected to an input end of the deep stimulation coil module;
the upper computer control software is configured to send a stimulation instruction to the system main control module according to predetermined parameter data, wherein the predetermined parameter data comprises a target region, excitation time, and an excitation current;
the system main control module is configured to send a control enable signal to the stimulation coil excitation power module when receiving the stimulation instruction;
the stimulation coil excitation power module is configured to provide a current to the deep stimulation coil module according to the control enable signal;
the deep stimulation coil module is configured to generate a superimposed magnetic field and transmit the superimposed magnetic field to the target region;
the deep stimulation coil module comprises a plurality of coil groups; each coil group comprises two coils; there is an overlapping part among magnetic fields of the plurality of coil groups, the overlapping part corresponds to a predetermined target region, and a current flowing through each coil group has same magnitude and direction, the plurality of coil groups are placed in different orientations on a brain;
a position and an angle of each coil group are adjusted according to the target region, such that electromagnetic fields generated by respective coil groups are superimposed in the target region:
by generating a superimposed electromagnetic field, a non-invasive transcranial magnetic field is able to accurately reach a deep brain region, wherein the deep brain region is a brain region with a depth greater than 6 cm, and the plurality of coil groups are added for greater intensity of regulatory stimulation; a use of the plurality of coil groups also reduces requirements on a coil excitation power supply, improves power usage efficiency, and reduces safety risks.

2. The device according to claim 1, further comprising a coil temperature detection module; wherein the coil temperature detection module is connected to the system main control module, and the coil temperature detection module is located inside the deep stimulation coil module; the coil temperature detection module is configured to collect a temperature of the deep stimulation coil module in real time, and to transmit the temperature to the system main control module; the system main control module is configured to control on/off of the stimulation coil excitation power module according to the temperature; the system main control module transmits the temperature to the upper computer control software, and the upper computer control software is configured to send a stop instruction to the system main control module when the temperature is greater than a predetermined temperature threshold, and the upper computer control software displays temperature failure information.

3. The device according to claim 1, further comprising a power failure detection module; wherein the power failure detection module is connected to the system main control module and the stimulation coil excitation power module; the power failure detection module is configured to collect an operating state of the stimulation coil excitation power module in real time, and to transmit the operating state to the system main control module; the system main control module is configured to control on/off of the stimulation coil excitation power module according to the operating state; the system main control module is further configured to transmit an off state of the stimulation coil excitation power module to the upper computer control software when controlling the stimulation coil excitation power module to be turned off; and the upper computer control software displays the off state of the stimulation coil excitation power module, and sends a stop instruction to the system main control module.
